(19) United States
(12) Patent Application Publication
(10) Pub. No.: US 2011/0098574 A1
(43) Pub. Date: Apr. 28, 2011

(54) PATIENT POSITION MONITORING APPARATUS

(76) Inventors: Dae Sung HWANG, Seoul (KR); Wan Cheol KIM, Hwasung-si (KR)

(21) Appl. No.: 12/605,058

(22) Filed: Oct. 23, 2009

(51) Int. Cl. A61B 6/04; A61B 5/11; A61B 8/00

(52) U.S. Cl. 600/473; 600/595; 600/437

(57) ABSTRACT

A patient position monitoring apparatus capable of monitoring the position of a patient is provided. The patient position monitoring apparatus includes a position guider emitting a source and a plurality of guide markers sensing the source emitted from the position guider and transmitting sensed information to the position guider. The position guider determines whether positions of the guide markers are changed through the sensed information. When the position of the patient is changed, and thus the source emitted from the position guider is out of the range of the guide markers, a warning signal is output. Accordingly, a user can monitor a variation in the position of the patient even if the user does not continuously observe the patient.

### BACKGROUND OF THE INVENTION

1. Field of the Invention

The present invention relates to a patient position monitoring apparatus, and more particularly, to a patient position monitoring apparatus capable of monitoring a variation in the position of a patient.

2. Discussion of the Related Art

There are many occasions when a patient should not be moved during medical treatment in a hospital because it is required to fix a body part of the patient to which the medical treatment is given. Accordingly, even a slight motion of the patient must be detected.

For example, ultrasonic therapy is required to radiate ultrasonic waves only to the affected part, and thus the motion of the patient must be continuously observed. Accordingly, a user (or a doctor) has to continuously watch the patient or marks the affected part of the patient, captures an image of the affected part using an optical camera and monitor the captured image in real time to detect a motion of the patient.

However, the aforementioned method requires the user to spend time because the user has to continuously monitor the patient. Furthermore, it is difficult to move the patient to the original position because the user determines the movement according to user's experience.

### SUMMARY OF THE INVENTION

It is an object of the present invention to provide a patient position monitoring apparatus which outputs a warning signal when a patient position is changed during medical treatment that requires the patient position to be fixed.

According to an aspect of the present invention, there is provided a position guider of a patient position monitoring apparatus, which includes a plurality of source emitting parts located above a patient to which a plurality of guide markers for sensing a source are attached and emitting the source to the guide markers; a first communication unit performing communication with the guide markers; and a first controller receiving information sensed by the guide markers from the guide markers through the first communication unit and determining whether the position of the patient is changed.

According to another aspect of the present invention, there is provided a guide marker of a patient position monitoring apparatus, which includes a source sensor sensing a source emitted from a position guider; a second communication unit performing communication with the position guider; and a second controller transmitting information sensed by the source sensor to the position guider through the second communication unit.

According to another aspect of the present invention, there is provided a patient position monitoring apparatus including a position guider emitting a source; and a plurality of guide markers sensing the source emitted from the position guider and transmitting sensed information to the position guider. The position guider determines whether positions of the guide makers are changed through the sensed information.

The patient position monitoring apparatus may further include a strap that has a first adhesive part for attaching the guide markers to the strap, which is formed on one side of the strap, and is located on the affected part of a patient. The first adhesive part may correspond to one of a Velcro tape and a metal fiber. A second adhesive part for attaching each guide marker to the strap may be formed on the bottom face of each guide marker, and the second adhesive part may correspond to one of a Velcro tape and a magnet.

The patient position monitoring apparatus may further include an output unit receiving a warning signal for warning a user that the positions of the guide markers are changed from the position guider and outputting the warning signal.

The position guider may judge that the position of the patient is changed if it is determined that the source emitted from the source emitting parts is out of the range of the source sensor through the sensed information.

According to the present invention, the guide markers are attached to portions to which a source is emitted from the source emitting pars, the guide markers respectively include the source sensor, and the position guider receives information sensed by the source sensor and outputs a warning signal when the source is out of the range of the source sensor to monitor a motion of the patient.

Furthermore, when the patient moves, the patient can be returned to the original position by moving the patient such that the source emitted from the source irradiating parts reach the range of the source sensor.

Moreover, the interval of the source irradiating parts can be previously determined, and thus a treatment route and treatment area for the patient can be calculated through the first controller included in the position guider to provide convenience and accuracy during medical treatment.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 illustrates a patient position monitoring apparatus according to an embodiment of the present invention;

FIG. 2 is a block diagram of ultrasonic therapy equipment including the patient position monitoring apparatus illustrated in FIG. 1;

FIG. 3 is a cross-sectional view of a strap and a guide marker of the patient position monitoring apparatus illustrated in FIG. 1 according to and embodiment of the present invention; and

FIGS. 4a, 4b, 4c and 4d illustrate various arrangements of source emitting parts of the patient position monitoring apparatus according to the present invention.

### DETAILED DESCRIPTION OF THE PREFERRED EMBODIMENTS

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals in the drawings denote like elements, and thus their description will be omitted.

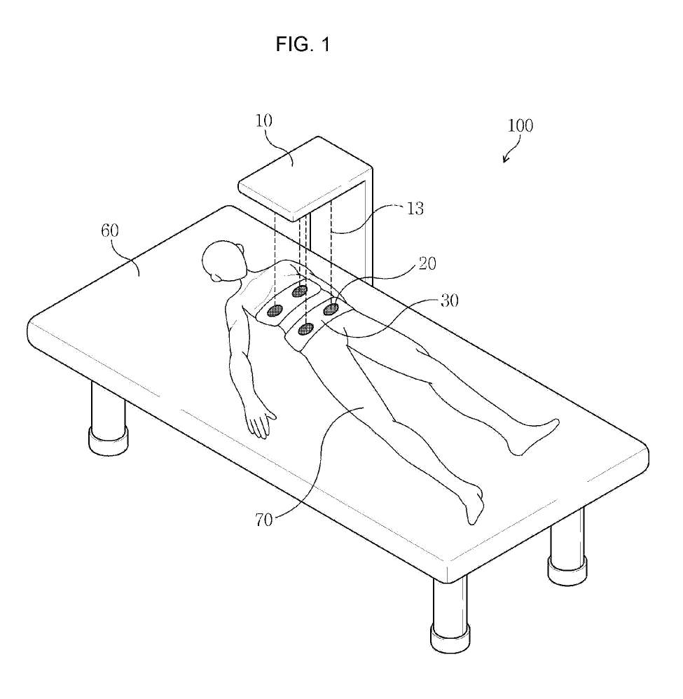

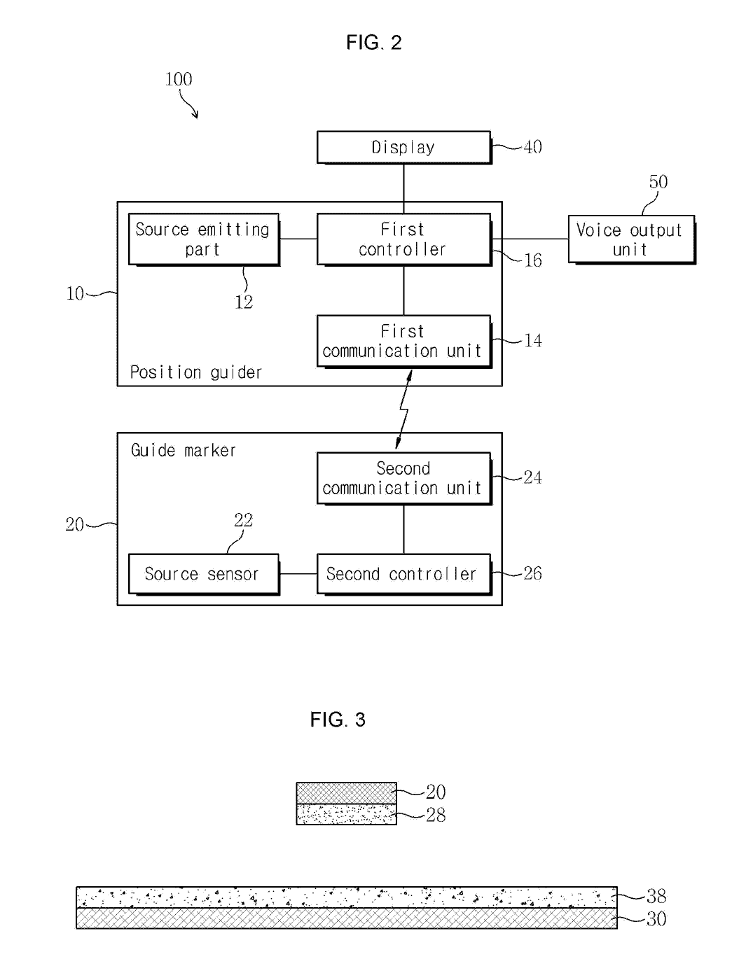

A patient position monitoring apparatus according to an embodiment of the present invention will now be explained with reference to FIGS. 1, 2 and 3. FIG. 1 illustrates the patient position monitoring apparatus 100 according to an embodiment of the present invention, FIG. 2 is a block diagram of ultrasonic therapy equipment including the patient position monitoring apparatus according to an embodiment of the present invention, and FIG. 3 is a cross-sectional view of a strap and a guide marker of the patient position monitoring apparatus according to the present invention.

The patient position monitoring apparatus 100 monitors the position of a patient 70 and outputs a warning signal if the position of the patient 70 is changed during medical treatment. The patient position monitoring apparatus 100 according to the current embodiment of the invention includes a position guider 10, a plurality of guide markers 20 and a strap 30. The patient position monitoring apparatus 100 may further include output units. Here, the output units include a display 40 and a voice output unit 50. The output units may further include a lamp which outputs a warning signal as flicking light.

The display 40 displays a menu of various functions executed in the patient position monitoring apparatus 100. The display 40 may indicate points to which the guide markers 20 are attached or a treatment route of the patient 70. A liquid crystal display (LCD) or a touch screen may be used as the display 40. The touch screen functions as both a display device and an input device.

The voice output unit 50 outputs a voice under the control of the position guider 10. Particularly, the voice output unit 50 receives the warning signal from the position guider 10 and outputs the warning signal as a voice.

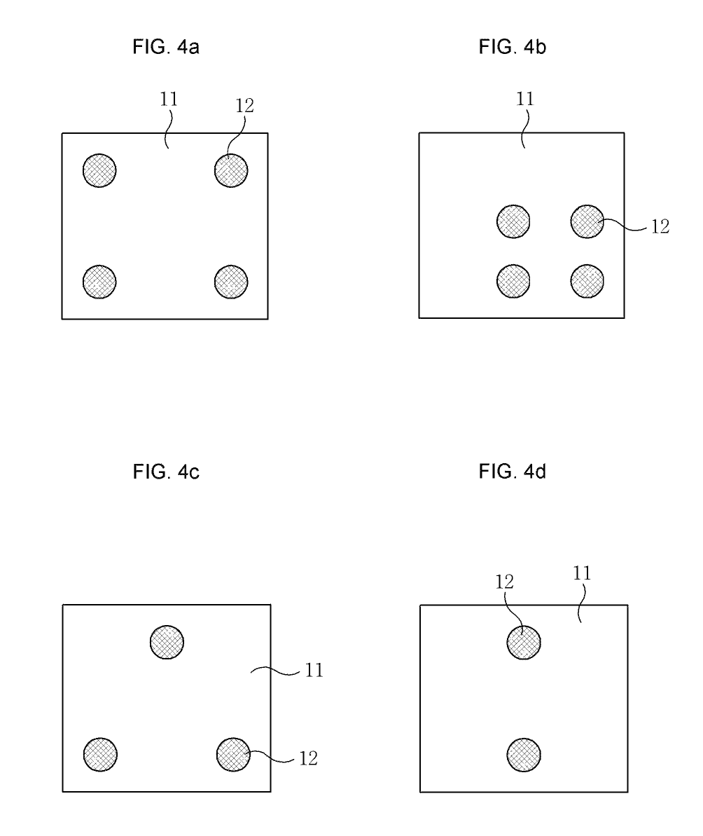

The position guider 10 includes source emitting parts 12, a first communication unit 14 and a first controller 16. The source emitting parts 12 are located above the patient to which the plurality of guide markers 20 are attached and emit a source to the guide markers 20. The number of source emitting parts 12 corresponds to the number of guide markers 20. The source emitting parts 12 are formed on a source emitting plate 11 (refer to FIG. 4) and emit one of a laser, LED light, ultrasonic waves, sprit light and infrared rays. For example, when the source emitting parts 12 emit laser beams 13, the laser beams 13 arrive at the patient 70 located under the source emitting parts 12 and are indicated on the strap 30 attached to the affected part of the patient 70. The source emitting parts 12 will be described later with reference to FIG. 4.

The first communication unit 14 performs communication with the guide markers 20. Here, wired or wireless communication can be used.

The first controller 16 corresponds to a microprocessor that controls the overall operation of the position guider 10. The first controller 16 receives information on the sensed source from the guide markers 20 and determines whether the position of the patient 70 is changed.

Further, the position guider 10 illustrated in FIG. 1 has the source emitting parts 12 formed on the side facing the patient 70. However, the shape of the position guider 10 is not limited thereto. The position guider 10 may be fixed to a therapy table 60 or a movable supporting table.

Each of the guide markers 20 includes a source sensor 22, a second communication unit 24 and a second controller 26. The source sensor 22 senses the source emitted from the corresponding source emitting part 12. The source sensor 22 may be placed opposite to the source emitting part 12 to stably sense the source emitted from the source emitting part 12. The source sensor 22 is selected according to the type of the source emitted from the source emitting part 12 and includes one of a laser sensor, an LED sensor, an ultrasonic sensor, a sprit light sensor and an infrared sensor. For example, when a laser is emitted from the source emitting part 12, the laser sensor is used as the source sensor 22.

The second communication unit 24 performs communication with the position guider 10. Here, wired or wireless communication may be used.

Further, the second controller 26 corresponds to a microprocessor which controls the overall operation of the guide markers 20. The second controller 26 transmits information sensed by the source sensor 22 to the position guider 10 through the second communication unit 24. The first communication unit 14 and the second communication unit 24 are connected to each other through a wired or wireless method.

The first controller 16 of the position guider 10 receives the information sensed by the source sensor 22, determines whether the source emitted from the source emitting part 12 is out of the range of the source sensor 22 through the received information and judges that the position of the patient 70 is changed if when the source is out of the range of the source sensor 22. When it is determined that the position of the patient 70 is changed, the first controller 16 can output a warning signal through the voice output unit 16. Further, the first controller 16 can output the warning signal through the display 40. When the position of the patient 70 is changed and thus the warning signal is output through the voice output unit 50 or the display 40, the user can recognize the change of the patient position and move the patient 70 to the original position such that the source irradiated from the source emitting part 12 reaches the range of the source sensor 22.

The strap 30 is used to attach the guide markers 20 to the patient 70 and is located on the affected part of the patient 70. The strap 30 includes a first adhesive part 38 formed on one side thereof. The strap 30 may cover the body of the patient 70 including the affected part or only the affected part. The first adhesive part 38 is formed to attach the guide markers 20 to the strap 30. A Velcro tape or a metal fiber can be used as the first adhesive part 38.

Each of the guide markers 20 includes a second adhesive part 28 formed on one side thereof. The second adhesive part 28 is attached to the first adhesive part 38 of the strap 30. The second adhesive part 28 may use a Velcro tape when the first adhesive part 38 is a Velcro tape and use a magnet when the first adhesive part 38 is a metal fiber. The first adhesive part 38 and the second adhesive part 28 are not limited to the Velcro tape, metal fiber and magnet and can use various methods which can attach the first and second adhesive parts 38 and 28 to each other.

Although the patient position monitoring apparatus 100 according to the current embodiment of the invention includes the display 40 and the voice output unit 50 which are separated from the position guider 10, the present invention is not limited thereto. For example, the position guider 10 may include the display 40 or the voice output unit 50.

The source emitting parts 12 according to an embodiment of the present invention will now be explained with reference to FIGS. 4a, 4b, 4c and 4d. FIGS. 4a, 4b, 4c and 4d illustrate various shapes of the source emitting parts of the patient position monitoring apparatus of the present invention.

The plurality of source emitting parts 12 are formed on the source emitting plate 11. The source emitting parts 12 are connected to the position guider 10 through a wired or wireless method and emit a source. The source emitting parts 12 may be respectively formed at four corners of the source emitting plate 11, as shown in FIG. 4a, and the interval or positions of the source emitting parts 12 may be varied, as shown in FIG. 4b. Further, the source emitting parts 12 may be arranged in a triangular shape, as shown in FIG. 4c, and two source emitting parts 12 may be formed in a row, as shown in FIG. 4d.

Though FIGS. 4a, 4b, 4c and 4d show the source emitting parts 12 in four arrangement forms, the present invention is not limited thereto.

Furthermore, the patient position monitoring apparatus 100 may include a cover for covering one of the plurality of source emitting parts 12 to reduce the number of source emitting parts 12.

The interval of the source emitting parts 12 formed on the source emitting plate 11 can be determined in advance, and thus the first controller 16 can calculate a treatment route or a treatment area for the patient through the previously determined interval of the sources 13 corresponding to the source emitting parts 12.

Moreover, the source emitting parts 12 may be designed such that the source emitting parts 12 can move on the source emitting plate 11. In addition, the source emitting parts 12 may be connected to a flexible rail without using the source emitting plate 11 to change the positions and number of the source emitting parts 12. The source emitting parts 12 can be formed in various manners.

While the present invention has been particularly shown and described with reference to exemplary embodiments thereof, it will be understood by those of ordinary skill in the art that various changes in forms and details may be made therein without departing from the spirit and scope of the present invention as defined by the following claims.

What is claimed is:

1. A position guider of a patient position monitoring apparatus, comprising:
a plurality of source emitting parts located above a patient to which a plurality of guide markers for sensing a source are attached and emitting the source to the guide markers;
a first communication unit performing communication with the guide markers; and
a first controller receiving information sensed by the guide markers from the guide markers through the first communication unit and determining whether the position of the patient is changed.

2. The position guider of a patient position monitoring apparatus of claim 1, further comprising a source emitting plate on which the plurality of source emitting parts are arranged.

3. The position guider of a patient position monitoring apparatus of claim 2, wherein the source emitting parts emit one of a laser, LED light, ultrasonic waves, sprit light and infrared rays.

4. A guide marker of a patient position monitoring apparatus, comprising:
a source sensor sensing a source emitted from a position guider;
a second communication unit performing communication with the position guider; and
a second controller transmitting information sensed by the source sensor to the position guider through the second communication unit.

5. The guide marker of a patient position monitoring apparatus of claim 4, wherein the source sensor includes one of a laser sensor, an LED sensor, an ultrasonic sensor, a sprit light sensor and an infrared sensor.

6. A patient position monitoring apparatus comprising:
a position guider emitting a source; and
a plurality of guide markers sensing the source emitted from the position guider and transmitting sensed information to the position guider,
wherein the position guider determines whether positions of the guide makers are changed through the sensed information.

7. The patient position monitoring apparatus of claim 6, further comprising a strap that has a first adhesive part for attaching the guide markers to the strap, which is formed on one side of the strap, and is located on the affected part of a patient.

8. The patient position monitoring apparatus of claim 7, wherein the first adhesive part corresponds to one of a Velcro tape and a metal fiber.

9. The patient position monitoring apparatus of claim 8, wherein a second adhesive part for attaching each guide marker to the strap is formed on the bottom face of each guide marker, and the second adhesive part corresponds to one of a Velcro tape and a magnet.

10. The patient position monitoring apparatus of claim 6, further comprising an output unit receiving a warning signal for warning a user that the positions of the guide markers are changed from the position guider and outputting the warning signal.

11. The patient position monitoring apparatus of claim 6, wherein the position guider comprises:
a plurality of source emitting parts located above a patient to which the plurality of guide markers are attached and emitting a source to the guide markers;
a first communication unit performing communication with the guide markers; and
a first controller receiving information sensed by the guide markers from the guide markers through the first communication unit and determining whether the position of the patient is changed.

12. The patient position monitoring apparatus of claim 11, wherein each of the guide markers comprises:
a source sensor sensing the source emitted from the position guider;
a second communication unit performing communication with the position guider; and
a second controller transmitting information sensed by the source sensor to the position guider through the second communication unit.

13. The patient position monitoring apparatus of claim 12, wherein the position guider judges that the position of the patient is changed if it is determined that the source emitted from the source emitting parts is out of the range of the source sensor through the sensed information.

14. The patient position monitoring apparatus of claim 13, further comprising an output unit receiving a warning signal for warning a user of a change in the patient position from the position guider and outputting the warning signal.
